(19) United States
(12) Patent Application Publication
(10) Pub. No.: US 2003/0004480 A1
(43) Pub. Date: Jan. 2, 2003

(54) Catamenial device change indicator

(76) Inventors: Anthony DiSalvo, Bernardsville, NJ (US); Raymond J. Hull JR., Hampton, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/991,124

(22) Filed: Nov. 16, 2001

Related U.S. Application Data
(60) Provisional application No. 60/302,084, filed on Jun. 29, 2001.

(51) Int. Cl. A61F 13/20

(52) U.S. Cl. 604/364; 604/385.18; 604/904

(57) ABSTRACT

An absorbent device for insertion into a vaginal cavity, the absorbent device having an absorbent body; and an indicator structure arranged and configured within the absorbent body, the indicator structure having a resilient member having a first arm having a rough surface and a second arm, the first and second arms being maintained in a strained configuration by a restraint; wherein the restraint weakens upon exposure to moisture and the resilient member is capable of articulating to a relaxed configuration upon the weakening of the restraint in a movement in which the rough surface of the first arm traverses the second arm to generate vibration discernible to the user.

### FIELD OF THE INVENTION

The present invention relates to tampons, and more particularly to a tampon wetness detection system that signals the user that the tampon is approaching absorbent capacity and it is time to change the tampon.

### BACKGROUND OF THE INVENTION

Tampons are designed to contain a particular amount of menstrual fluid. The amount of menstrual fluid absorbed by a tampon can vary depending on absorbency levels. For example, in the United States, tampon absorbency can range from less than 6 grams (Junior absorbency) grams to 15-18 grams (Ultra absorbency). In order to ascertain whether a tampon has reached its absorbent capacity, the tampon must be removed and viewed, resulting in the destruction of the tampon, as most women are reluctant to reinsert the tampon. In most cases, a user will remove a tampon before it has reached its absorbent capacity in order to prevent an accident wherein the absorbent capacity of the tampon is exceeded. Once the absorbent capacity is exceeded, the excess menses flows unimpeded from the vagina to soil the user's clothing.

A determinative criteria frequently used to gauge tampon replacement is the amount of time elapsed since insertion. The time elapsed criteria for changing tampons is not satisfactory for several reasons, e.g., the menstrual flow rate varies throughout the menstruating period and much adsorbent capacity of tampons is wasted due to the tendency to change before an accident occurs.

The flow variation throughout the period causes problems as to how long to wear a tampon because a user cannot establish a definite time period for which the absorbent capacity within a tampon is sufficient. Therefore, she is in a quandary as to how long to wear specific tampons during days of heavy flow as contrasted to days of light flow.

A correlation between tampon performance during light flow versus heavy flow is difficult for the user to make. Thus, since the user would rather be safe than sorry, she frequently removes a tampon before the absorbent capacity of the tampon has been reached and wastes much of the product she had purchased.

Tampons have been made larger and with different materials to obtain higher absorbencies, often resulting in product claims that a user would not have to change the tampon as often. But the user would still waste a portion of the tampon absorbent capacity, as most users are not willing to risk having an accident. Therefore, bigger tampons provide a longer wearing time but do not approach the problem of fully using the absorbent capacity within a tampon. In rare cases, should a user leave the tampon in for an extended period of time, a life threatening infection may develop.

Wearing a high absorbency tampon can lead to discomfort and other problems as well. Women will sometimes wear a larger absorbency tampon due to the fear of tampon failure, especially if she is uncertain how often she will be able to access privacy in order to change the tampon. If the tampon is unsaturated, there may be drying of the vaginal wall, which may cause discomfort upon the tampon's removal.

Thus, there is a need for a tampon wetness detection system that signals the user to change the tampon prior to soiling the user's clothing.

### SUMMARY OF THE INVENTION

An absorbent device for insertion into a vaginal cavity, the absorbent device having an absorbent body; and an indicator structure arranged and configured within the absorbent body, the indicator structure having a resilient member having a first arm having a rough surface and a second arm, the first and second arms being maintained in a strained configuration by a restraint; wherein the restraint weakens upon exposure to moisture and the resilient member is capable of articulating to a relaxed configuration upon the weakening of the restraint in a movement in which the rough surface of the first arm traverses the second arm to generate vibration discernible to the user.

### BRIEF DESCRIPTION OF THE DRAWING

FIG. 1 is a plan view of the resilient member opened and flattened according to one embodiment of the invention.

FIG. 2 is a plan view of resilient member of FIG. 1 in an unstrained configuration according to one embodiment of the invention.

FIG. 3 is a plan view of the resilient member of FIG. 2 in a strained configuration.

FIG. 4 is a plan view of the resilient member of FIG. 1 in an unstrained configuration according to another embodiment of the invention.

FIG. 5 is a plan view of the resilient member of FIG. 4 in a strained configuration.

FIG. 6 is a plan view of the resilient member of FIG. 4 in a more strained configuration.

FIG. 7 is a longitudinal cross-section of a tampon having the resilient member of FIG. 6.

FIG. 8 is a longitudinal cross-section view of a tampon having the resilient member in an unstrained, relaxed configuration.

### DETAILED DESCRIPTION OF THE PREFERRED EMBODIMENTS

As used herein the specification and the claims, the term “resilience” and variants thereof describes the capability of a strained body to recover its size and shape after deformation caused especially by bending, compressing, twisting, stretching or any combination thereof A resilient member can go from a relaxed configuration to a strained configuration to a relaxed configuration any number of times without losing substantially the ability to recover its original shape. A resilient member in a strained configuration possesses strain energy or potential energy of deformation. In the present invention, the release of the stored potential energy may cause the resilient member to revert to the relaxed configuration, resulting in a potential for kinetic movement—movement of portions of the resilient member within the catamenial device. The user may feel a discernible vaginal tactile sensation caused by this movement or motion.

As used herein the specification and the claims, the term “weaken” and variants thereof describe the loss of strength and/or integrity of a material, especially upon exposure to fluid or moisture. The material may loose its cohesive or adhesive nature, swell, dissolve or simply weaken such that it no longer can perform as in a dry state. Thus, the material no longer has the strength to restrain the resilient member in a strained configuration.

In general, the absorbent article of this invention has at least two parts: a catamenial absorbent device and a wetness indicating structure. The wetness indicating structure may be a single unit or may have multiple components. The catamenial device may be a tampon and can also be either a tampon used with an applicator or one that is inserted digitally.

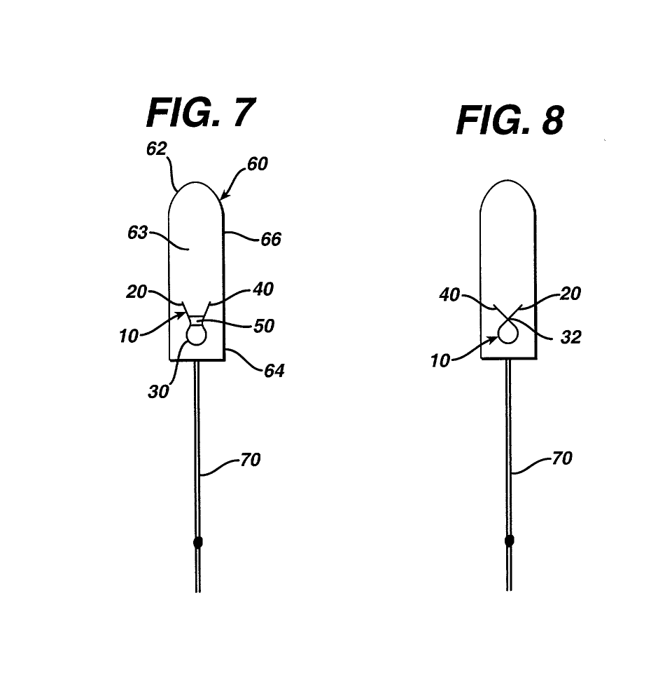

In the present invention, tampon 60 (tampon configuration shown in FIGS. 7 and 8) has an absorbent core 63, resilient member 10, an upper portion 62, a lower portion 64, and may optionally include string 70 and cover 66 covering absorbent core 63. Resilient member 10 may be a spring or any other shaped device that may go from a natural, relaxed configuration into a strained or compressed configuration and back to the relaxed configuration. The strained configuration of resilient member 10 is maintained by restraint 50. In the present embodiment, resilient member 10 is held and maintained in a compressed configuration by restraint 50 while the tampon is in a dry state. Upon penetration of fluid into the tampon, restraint 50 weakens, which allows resilient member 10 to assume its original, more relaxed configuration. This relaxation, which occurs as a quick release, produces a vibration or sensory movement that leads to a discernible sensation.

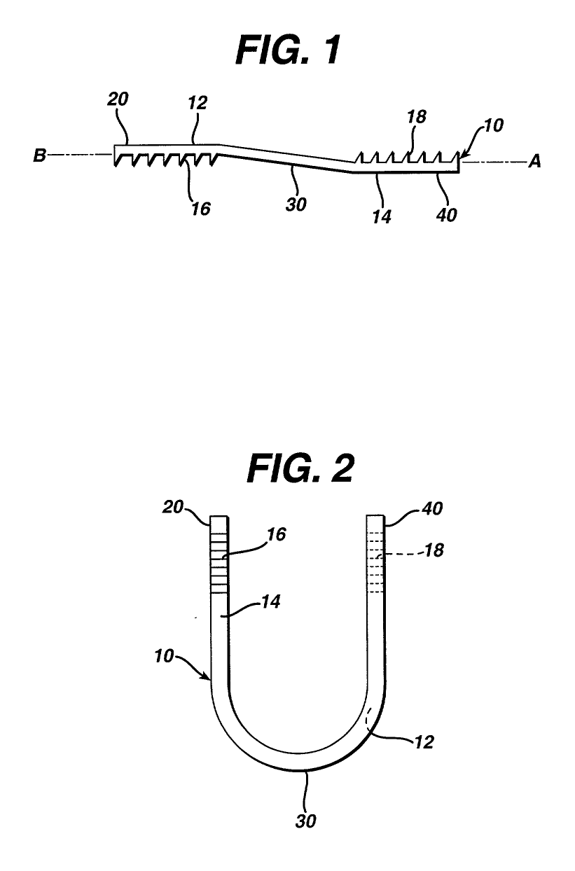

FIG. 1 shows resilient member 10 in top plan view. Resilient member 10 has first arm 20, second arm 40, central portion 30, first surface 12 and second surface 14. First surface 12 and second surface 18 are on opposing surfaces. At least one surface of an arm is roughened. For example, the first arm has a rough surface 16. Additionally but not necessarily, second arm 40 may have a rough surface 18, too. As shown in FIG. 1, both arms have rough surfaces 16 and 18, which may be in the form of ridges, teeth, bumps, undulations, serrations or other surface irregularities (in a uniform or random pattern) that cause a vibration when rubbed against each other. First arm 20, second arm 40 and central portion 30 are aligned such that rough surfaces 16 and 18 are in the same plane as indicated by dotted line “AB” (shown FIG. 1).

While FIG. 1 shows two rough surfaces 16 and 18, a single rough surface may be sufficient to cause vibrations or a scrapping sensation when rubbed or traversed across or against a smooth or smoother surface. In the following examples, it is understood that only one surface need have a rough surface.

As previously mentioned, the resilient member may be made from any resilient material capable of undergoing a deformation without loosing the ability to recover its original shape. A non-limiting list of these materials include elastomers and plastics such as polyacetals, polyolefins (e.g., polyethylenes and polypropylenes), nylons, rubbers, polyurethanes; and metals such as copper, stainless steel, spring steel, titanium, nickel, nitinol, and metals coated with any type of non-corrosive coating such as elastomers or plastics.

Resilient member 10 may be placed anywhere in the tampon but it is preferable that it be located near to lower portion 64 of tampon 60. While not being bound by any particular theory, it is believed that the vagina has a sensitive region surrounding the introitus and would be more receptive to the tactile movement of the wetness indicator.

FIG. 2 shows a plan view of resilient member 10 in an unstrained configuration. First arm 20 has rough surface 16. Second arm 40 may have rough surface 18 (ridges not visible but represented by dotted lines). First arm 20 and second arm 40 are connected to central portion 30. First arm 20 and second arm 40 do not contact in the unstrained configuration.

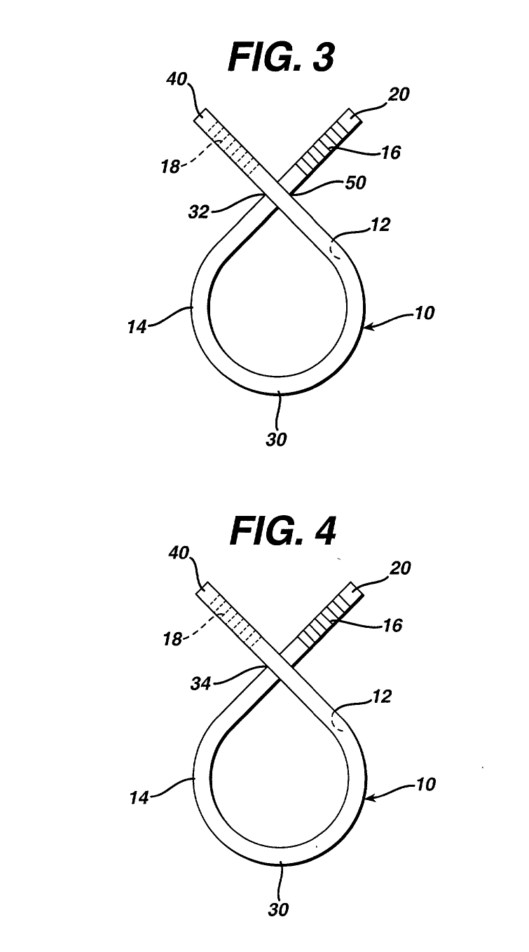

FIG. 3 shows resilient member 10 of FIG. 2 in a strained configuration. Second arm 40 crosses over first arm 20 such that first surface 12 contacts second surface 14 at point of contact 32. Restraint 50 holds resilient member 10 in the strained configuration. Upon use in a tampon, fluid eventually penetrates and begins to weaken restraining member 50. When restraint 50 is sufficiently weakened, first surface 12 looses contact with second surface 14 at point of contact 32. As rough surfaces 16 and 18 are in the same plane (shown dotted line AB in FIG. 1), rough surface 16 and 18, upon the weakening of restraining member 50, traverse or scrape against each other, producing a vibrating sensation that the user can detect. As previously mentioned, it is not necessary for both first surface 12 and second surface 14 to have rough surfaces.

The tie or restraint can be made from any soluble, water swellable material or any material that weakens or looses integrity upon exposure to fluid or moisture. These materials include but are not limited to gelatins, water soluble adhesives, cellulose derivatives including HPMC (hydroxypropyl methyl cellulose) and ethyl cellulose, polyvinyl alcohol, polyether urethane, polyethylene oxide, polyacrylamide and copolymers thereof, and polyacrylic acid. Materials that loose the ability to form cohesive bonds upon exposure to fluid may also be used. The restraint may be in the form of a ribbon, a band or a drop that sets and forms a bond, as in the case of an adhesive.

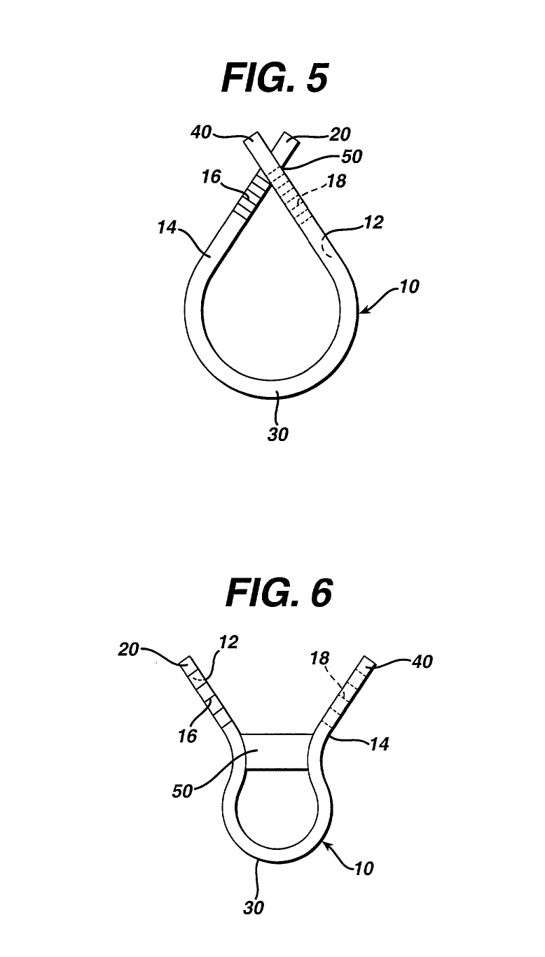

FIGS. 4-5 show another embodiment of the invention. In this embodiment, the unstrained configuration is shown in FIG. 4. Second arm 40 crosses over first arm 20 such that first surface 12 contacts second surface 14 at overlay 34. FIG. 5 shows the resilient member 20 of FIG. 4 in a strained configuration. Point of contact 36 between arm 20 and arm 40 is maintained by restraint 50. Upon use, restraint 50 weakens and resilient member 10 begins to revert into the relaxed configuration of FIG. 4. During this relaxation process, rough surface 16 scrapes or traverses against rough surface 18, causing a vibration or other detectable sensation. Again, as previously mentioned, it is not necessary for both first surface 12 and second surface 14 to have rough surfaces.

FIG. 6 shows resilient member 10 held in a strained configuration by restraint 50. As shown, restraint 50 holds resilient member 10 open such that first arm 21 and second arm 40 do not cross over in the strained configuration.

FIG. 7 shows a cross section of tampon 60 containing resilient member 10 in a strained configuration. As shown, first arm 20 and second arm 40 are held apart by restraint 50. In one embodiment, first arm 20 and second arm 40 are positioned toward insertion end 62 of tampon 60. In another embodiment, central portion 30 is positioned toward insertion end 62 of tampon 60. As seen in FIG. 7, tampon 60 may include removal string 70 and cover 66. Removal string 70 may be attached by any known means known in the art. In one embodiment, string 70 is attached to lower portion 64 of tampon 60. In another embodiment, string 70 is attached to central portion 30 of resilient member 10.

FIG. 8 shows resilient member 10 is an unstrained configuration. Upon exposure to fluid, restraint 50 softens and weakens to the point where the potential energy of the resilient member 10 overcomes the force exerted by restraint 50 to keep first arm 20 and second arm 40 from crossing over. As the two arms traverse, rough surfaces 16 and 18 momentarily contact, causing a vibration or scraping sensation. This vibration or scraping is discernible to the user.

Detection by the wearer of the wetness indicator is preferably subtle enough not to startle the user. It is preferred that the resilient member have a quick release or expansion from the strained state to the relaxed state in a fashion to be noticeable. A slow expansion may be less discernible to the wearer.

Absorbent tampons are usually substantially cylindrical masses of compressed absorbent material having a central axis and a radius that defines the outer circumferential surface of the tampon. Tampons are often formed by first obtaining a shaped mass of absorbent material called a tampon blank. This blank can be in the form of a roll of sheet-like material, a segment of a continuous absorbent material, a mass of randomly or substantially uniformly oriented absorbent material, an individually prepared or cast mass of absorbent material, and the like.

The tampon blank is relatively uncompressed and has a relatively low density. It may be compressed to form a product having overall dimensions less than those of the blank prior to use. The compressed tampons may have a generally uniform density throughout the tampon, or they may have regions of differing density as described in the commonly assigned applications to Friese et al., U.S. Ser. No. 07/596,454, and Leutwyler et al., U.S. Pat. No. 5,813,102, the disclosures of which are herein incorporated by reference. Tampons also usually include a cover or some other surface treatment and a withdrawal string or other removal mechanism.

It is preferred that the wetness indicator of the present invention be contained within the central portion of the tampon.

Another type of tampon that may contain a wetness indicating device may be the bag or sack-type tampon. In this type of tampon, absorbent material is contained within a overwrap that is at least partially fluid permeable. Examples of bag-type tampons are disclosed in U.S. Ser. No. 09/741,718 (Buzot), U.S. Ser. No. 09/823,045 (Buzot) and U.S. Ser. No. 09/874,451 (Intravartolo et al.), the entire contents which are hereby incorporated by reference.

Absorbent material useful in tampon formation includes fiber, foam, superabsorbents, hydrogels, and the like. Preferred absorbent material for the present invention includes fiber and foam.

Preferably, the fibers include hydrophilic fibers, and more preferably, the fibers include absorbent fibers, i.e., the individual fibers, themselves, absorb fluid. A useful, non-limiting list of useful tampon fibers includes natural fibers such as cotton, wood pulp, jute, and the like; and processed fibers such as regenerated cellulose, cellulose nitrate, cellulose acetate, rayon, polyester, polyvinyl alcohol, polyolefin, polyamine, polyamide, polyacrylonitrile, and the like. Other fibers in addition to the above fibers may be included to add desirable characteristics to the absorbent body. For example, hydrophobic fibers may be used in outer surfaces of the tampon to reduce surface wetness and hydrophilic fibers may be used to increase the rate of fluid transport into and throughout the body. Preferably, the tampon fibers are rayon or cotton, and more preferably, the fibers are rayon. The fibers may have any useful cross-section.

Preferred fiber cross-sections include multi-limbed and non-limbed. More preferably, the fibers are predominantly multi-limbed. Multi-limbed, regenerated cellulosic fibers have been commercially available for a number of years. These fibers are known to possess increased specific absorbency over non-limbed fibers. One commercial example of these fibers are the Danufil VY multilimbed viscose rayon fibers available from Acordis Ltd., London, England. These fibers are described in detail in Wilkes et al., U.S. Pat. No. 5,458,835, the disclosure of which is hereby incorporated by reference.

Preferably, the foams include hydrophilic foams, and more preferably, the foams may include absorbent foams, i.e., the foam cells, themselves, absorb fluid.

A fluid-permeable cover may substantially enclose the tampon blank. Thus, the cover encloses a majority of the outer surface of the tampon. This may be achieved as disclosed in Friese, U.S. Pat. No. 4,816,100, the disclosure of which is herein incorporated by reference. In addition, the cover may enclose either or both ends of the tampon. Of course, for processing or other reasons, some portions of the surface of the tampon may be free of the cover. For example, the insertion end of the tampon and a portion of the cylindrical surface adjacent this end may be exposed, without the cover to allow the tampon to more readily accept fluids.

The cover can ease the insertion of the tampon into the body cavity and can reduce the possibility of fibers being separated from the tampon. Useful covers are known to those of ordinary skill in the art. They may be selected from an outer layer of fibers which are fused together (such as by thermobonding), a nonwoven fabric, an apertured film, or the like. Preferably, the cover has a hydrophobic finish.

The wetness indicating device can be inserted into the tampon blank prior to compression when the winding mandrel first rolls the nonwoven ribbon into an essentially cylindrical form. The wetness indicating device can also be inserted into the voids left when the winding mandrel is removed from the cylindrical form. Alternately, the wetness indicating device can be inserted after compression.

If other materials such as foam are used to make the tampon, the resilient member may be inserted through a slit cut into the lower portion of the tampon. Additionally, if the tampon has a cover, the resilient member can be placed between the cover and absorbent material, encircling the absorbent material. This may indent the tampon.

Tampons are generally categorized in two classes: applicator tampons and digital tampons. Applicator tampons use a relatively rigid device to contain and protect the tampon prior to use. To insert the tampon into a body cavity, the applicator is partially inserted into the body cavity, and the tampon can be expelled therefrom. Because the rigid applicator device protects the tampon, the tampon need not have a high degree of dimensional stability. In contrast, digital tampons do not have an applicator to help guide them into the body cavity and require sufficient stability to allow insertion without using an applicator.

We claim:

1. An absorbent device for insertion into a vaginal cavity, the absorbent device comprising:
a) an absorbent body; and
b) an indicator structure arranged and configured within the absorbent body, the indicator structure comprising a resilient member having a first arm having a rough surface and a second arm, the first and second arms being maintained in a strained configuration by a restraint;
wherein the restraint weakens upon exposure to moisture and the resilient member is capable of articulating to a relaxed configuration upon the weakening of the restraint in a movement in which the rough surface of the first arm traverses the second arm to generate vibration discernible to the user.

2. The absorbent device of claim 1 wherein the absorbent body comprises absorbent material.

3. The absorbent device of claim 2 wherein the absorbent material is formed of fibers, foams, or combinations thereof.

4. The absorbent device of claim 1 wherein the restraint maintains the resilient member in a stretched configuration.

5. The absorbent device of claim 1 wherein the restraint maintains the resilient member in a bent configuration.

6. The absorbent device of claim 1 wherein the restraint maintains the resilient member in a twisted configuration.

7. The absorbent device of claim 1 wherein the first arm is maintained across the second arm.

8. The absorbent device of claim 1 wherein the first arm is maintained in a spaced relationship from the second arm.

9. The absorbent device of claim 1 wherein the second arm has a rough surface.

10. The absorbent device of claim 1 wherein the resilient member is formed of plastics, metals, and combinations thereof.

11. The absorbent device of claim 1 wherein the restraint comprises a water soluble material.

12. The absorbent device of claim 11 wherein the restraint comprises a material selected from the group consisting of gelatins, water soluble adhesives, cellulose derivatives including HPMC (hydroxypropyl methyl cellulose) and ethyl cellulose, polyvinyl alcohol, polyether urethane, polyethylene oxide, polyacrylamide and copolymers thereof, polyacrylic acid and combinations thereof.

13. An indicator device comprising a resilient member having a first arm having a rough surface and a second arm, the first and second arms being maintained in a strained configuration by a restraint wherein the restraint weakens upon exposure to moisture and the resilient member is capable of articulating to a relaxed configuration upon the weakening of the restraint in a movement in which the rough surface of the first arm traverses the second arm to generate vibration.

14. A method of absorbing aqueous vaginal fluids comprising the steps of:
a) inserting an absorbent device into a vaginal cavity, the absorbent device comprising an absorbent body and an indicator structure arranged and configured within the absorbent body, the indicator structure comprising a resilient member having a first arm having a rough surface and a second arm, the first and second arms being maintained in a strained configuration by a restraint, wherein the restraint weakens upon exposure to moisture and the resilient member is capable of articulating to a relaxed configuration upon the weakening of the restraint in a movement in which the rough surface of the first arm traverses the second arm to generate vibration discernible within the vaginal cavity;
b) allowing the absorbent device to absorb sufficient aqueous vaginal fluids to weaken the restraint and to permit the resilient member to revert to the relaxed configuration;
c) detecting the vibrations generated by the first arm traversing the second arm; and
d) removing the absorbent device from the vaginal cavity.

15. The method of claim 14 further comprising the step of inserting a second absorbent device into the vaginal cavity after the step of removing the absorbent device from the vaginal cavity.

16. A method of making an absorbent device comprising the steps of:
a) manipulating a resilient member having a first arm having a rough surface and a second arm into a strained configuration;
b) applying a restraint to maintain the resilient member in the strained configuration, forming an indicator structure, the restraint being capable of weakening upon exposure to moisture and the resilient member is capable of articulating to a relaxed configuration upon the weakening of the restraint in a movement in which the rough surface of the first arm traverses the second arm to generate vibration discernible to a user; and
c) forming an absorbent body containing the indicator structure.

17. The method of claim 16 wherein the step of forming the absorbent body comprises forming the absorbent body around the indicator structure.

18. The method of claim 16 wherein the step of forming the absorbent body comprises forming the absorbent body and inserting the indicator structure into the absorbent body.
